(19) United States
(12) Patent Application Publication
(10) Pub. No.: US 2025/0000575 A1
(43) Pub. Date: Jan. 2, 2025

(54) TISSUE CUTTING SYSTEMS AND METHODS

(71) Applicants: The United States of America, as Represented by the Secretary, Department of Health and Human Srvcs., Bethesda, MD (US); Transmural Systems LLC, Andover, MA (US)

(72) Inventors: Robert J. LEDERMAN, Bethesda, MD (US); Toby ROGERS, Bethesda, MD (US); Jaffar M. KHAN, Bethesda, MD (US); Christopher G. BRUCE, Bethesda, MD (US); Adam GREENBAUM, Andover, MA (US); Vasilis BABALIAROS, Andover, MA (US)

(21) Appl. No.: 18/763,517

(22) Filed: Jul. 3, 2024

Related U.S. Application Data
(63) Continuation of application No. PCT/US2023/060223, filed on Jan. 6, 2023.
(60) Provisional application No. 63/297,226, filed on Jan. 6, 2022.
(60) Provisional application No. 63/307,939, filed on Feb. 8, 2022.

(51) Int. Cl. A61B 18/14; A61B 18/00; A61B 18/12; A61B 90/00

(52) U.S. Cl. CPC A61B 18/1492; A61B 18/1206; A61B 90/39; A61B 2018/00083; A61B 2018/00107; A61B 2018/00369; A61B 2018/00601; A61B 2018/126; A61B 2018/144; A61B 2090/3966

(57) ABSTRACT

The disclosure provides various embodiments of systems to facilitate the cutting of myocardial tissue structures percutaneously.

### CROSS-REFERENCE TO RELATED APPLICATION

The present patent application is a continuation of and claims the benefit of priority to International Patent Application No. PCT/US2023/060223, filed Jan. 6, 2023, which in turn claims the benefit of priority to U.S. Patent Application No. 63/297,226, filed Jan. 6, 2022, and U.S. Patent Application No. 63/307,939, filed Feb. 8, 2022. Each of the aforementioned patent applications is incorporated by reference herein in its entirety for all purposes.

### BACKGROUND

The disclosure relates generally to medical treatment devices and techniques, and, in some aspects, to methods and devices for diagnosis and treatment of myocardial tissue. The present disclosure provides improvements over the state of the art.

### SUMMARY OF THE DISCLOSURE

BASILICA and LAMPOON are aortic and mitral leaflet laceration procedures that use transcatheter electrosurgery. A guidewire traverses potentially obstructive heart valve leaflet tissue and then the inner-curvature of the kinked guidewire traversing the leaflet is electrified during traction to accomplish a longitudinal split of the leaflet.

Left ventricular outflow tract (LVOT) obstruction complicates hypertrophic cardiomyopathy and transcatheter mitral valve replacement. Septal reduction therapies including surgical myectomy and alcohol septal ablation are limited by surgical morbidity or coronary anatomy and high pacemaker rates respectively. Applicants have developed a novel transcatheter procedure, mimicking surgical myotomy, called SESAME (SEptal Scoring Along the Midline Endocardium). The SESAME procedure uses an insulation-modified guidewire to lacerate myocardium (heart muscle) instead of heart leaflet tissue, using a different system design from the BASILICA and LAMPOON procedures. In some aspects, the SESAME electrosurgical procedure can include an asymmetric insulation gap astride the guidewire kink, or bend. The kink or bend concentrates electrical charge and helps to position the charge-delivery-device at the therapy target to avoid bystander injury. The insulation gap, discussed below, is intended to overcome the tendency of charge to concentrate on the outer aspect of a kink.

This can be accomplished with the electrosurgical guidewire systems set forth herein. This permits safe and simple scoring of myocardial tissue, such as obstructive myocardial tissue, to relieve left ventricular outflow tract obstruction in hypertrophic cardiomyopathy. In a further aspect, this can also be applied to increase the compliance of stiff myocardium that causes heart failure with preserved ejection fraction (HFpEF), sometimes described as diastolic heart failure, which accounts for approximately half of heart failure hospital admissions. HFpEF can arise from multiple causes, such as hypertensive cardiomyopathy and infiltrative cardiomyopathy such as from amyloid.

Thus, in accordance with some aspects of the present disclosure, an electrosurgical guidewire is provided that includes a core wire having a proximal end, and a distal end and being defined by an outer surface between the proximal end and the distal end of the core wire. The core wire has a centerline that traverses the length of the core wire from the proximal end to the distal end of the core wire. In some implementations, a dielectric coating can be disposed about the core wire, wherein the proximal end and distal end of the core wire are exposed and the proximal end is configured to be coupled to an electrosurgical generator. The electrosurgical guidewire further includes a kink formed into the guidewire. The guidewire further defines an electrically exposed region. The electrically exposed region can be defined by a portion of the dielectric coating being absent from, or having been physically removed from (or not applied to, or masked from) the surface of the core wire along an inner surface of one side of the kink, with the dielectric coating being left intact on an outer surface and an opposing inner surface of the kink, or the electrically exposed region can be present on both sides of the kink.

In further accordance with the disclosure, implementations of an electrosurgical system are provided, including an electrosurgical generator, and an electrosurgical guidewire as disclosed herein coupled to the electrosurgical generator. The electrically exposed region of the guide wire can be disposed on only one side of the kink, and the electrosurgical generator can be electrically coupled to one end of the guidewire and to the patient, wherein the electrosurgical system operates in a monopolar mode. In a further implementation, an electrosurgical system is provided that includes an electrosurgical generator, and an electrosurgical guidewire as disclosed herein coupled to the electrosurgical generator. The electrically exposed region can be disposed on both sides of the kink, and the electrosurgical generator can be electrically coupled to both ends of the guidewire, wherein the electrosurgical system operates in a bipolar mode, and the current flows across tissue disposed in the kink during operation of the electrosurgical system.

In accordance with further aspects of the electrosurgical system, the electrically exposed region can be defined by two discrete exposed areas separated by a fully insulated length of the guidewire in the region of the kink. If desired, the electrically exposed region can define at least one raised surface thereon to concentrate the electric field. In some implementations, the electrically exposed region can include a radiopaque electrically conductive coating. The radiopaque electrically conductive coating can include a metal or alloy of high electrical conductivity. If desired, the radiopaque electrically conductive coating can include gold.

While certain embodiments herein begin with a straight, insulated guidewire that is kinked and denuded to create the electrically exposed region, the charge concentration device can alternatively be provided in a pre-manufactured format, such as where the kink is preformed into the guidewire prior to the guidewire being insulated. In such implementations, the region to be electrically exposed can be masked such that the insulation is not applied to that area. This can have the additional advantage of a radiopaque coating in the region of the kink remaining intact since the core wire does not go through a stripping or scraping step. This can enhance visibility under visualization, and it is possible that the enhanced conductivity of the radiopaque (e.g., gold) coating can act to enhance current flow and/or charge concentration. If desired, gold, for example, can be formed into the core wire by way of a plunge grinding technique as discussed elsewhere herein to concentrate gold in the region of charge concentration to affect the flow of electrical current when cutting tissue.

In accordance with further aspects, the electrosurgical guidewire can further include a radiopaque marker disposed over the core wire wherein the guidewire is formed into the kink proximate, adjacent, or at least partially within the radiopaque marker. The dielectric coating can be formed over the radiopaque marker. The electrically exposed region can be further defined by a portion of the radiopaque marker pattern having been physically removed from the surface of the core wire along the inner surface of one side of the kink, with the radiopaque marker and dielectric coating being left intact on the outer surface and the opposing inner surface of the kink.

In accordance with further aspects, the kink can have a radius of curvature between about 1 mm and about 7 mm, between about 2 mm and about 5 mm, between about 3 mm and about 4 mm or any increment therebetween of 0.5 mm in any of the aforementioned ranges. The kink can be “V”-shaped with a sharp bend and relatively short radius of curvature, or can be more “U”-shaped with a relatively larger radius of curvature. In some implementations, the electrically exposed region can have a length between about 1 mm and about 20 mm, about 3 mm and about 6 mm, or about 4 mm and about 8 mm, for example, or any increment therebetween of 0.5 mm in any of the aforementioned ranges. The radiopaque marker is useful to help position the region of charge concentration (uninsulated area) proximate tissue to be ablated. The shape and dimensions of the kink effect to disperse ablative energy over a desired (e.g., narrower or wider) target area.

In some implementations, the radiopaque marker can be formed at least in part from a radiopaque metallic material, preferably a biocompatible metal, deposited over the core wire, such as gold, platinum, or the like. The radiopaque marker can include an uneven surface configured to enhance its visual signature under fluoroscopy.

In accordance with further aspects, the electrosurgical guidewire can have an outer diameter of about 0.014 inches, for example. The electrosurgical guidewire can further include a radiopaque coil surrounding the distal tip of the guidewire between about 0.5 cm and 4 cm in length, for example. The electrically exposed region of the guidewire can be located distally with respect to the kink of the guidewire.

The present disclosure further provides implementations of an electrosurgical system that includes an electrosurgical generator, and an electrosurgical guidewire as set forth herein coupled to the electrosurgical generator. The system can further include a first proximal support catheter disposed over the electrosurgical guidewire between the kink and the proximal end of the electrosurgical guidewire. The system can further include a first distal support catheter disposed over the electrosurgical guidewire between the kink and the distal end of the electrosurgical guidewire. If desired, the system can further include a second proximal support catheter disposed over the electrosurgical guidewire and underneath the first proximal support catheter between the kink and the proximal end of the electrosurgical guidewire to provide additional columnar support to the system.

The system also provides implementations of a method of performing a myocardial tissue cutting procedure. The method can include directing a distal end of an electrosurgical guidewire as described elsewhere herein into the patient's vasculature through a passageway defined through myocardial tissue. The method can further include capturing the distal end of the electrosurgical guidewire with a snare catheter. The method can further include pulling the distal end of the electrosurgical guidewire out of the patient to externalize it alongside a proximal region of the electrosurgical guidewire. The method can still further include advancing the electrosurgical guidewire until the electrically exposed region is in contact with the myocardial tissue within the passageway. The method can further include electrifying the electrosurgical guidewire, and cutting the myocardial tissue by pulling the electrosurgical guidewire through the myocardial tissue.

In some implementations, the method can further include directing at least one supporting catheter over a proximal portion of the externalized guidewire and a distal portion of the externalized guidewire until a distal tip of each said supporting catheter is located proximate the kink in the guidewire. The supporting catheter is preferably configured to be made at least in part from an electrically insulating material. This can have the effect of protecting non-target tissue from injury, such as the aortic valve and leaflets during a SESAME procedure. It will be appreciated that such insulation can be located on catheters as desired to minimize electrical leakage from the core wire to both protect tissue and more efficiently direct ablative energy to target tissue.

In some implementations, the myocardial tissue can include a left ventricular outflow tract (LVOT) that is excessively narrow, due to one or more of a variety of causes. This can be caused by an obstruction, and/or simply the local anatomy. Sometimes, patients do not have a LVOT obstruction until after they have received implantation of a prosthetic mitral valve. Regardless, cutting the myocardial tissue can enlarge the LVOT. In some implementations, the myocardial tissue can include ventricular tissue, and cutting the myocardial tissue can enhance relaxation in hypertrophied myocardium. In some implementations, the system can be operated in a monopolar configuration wherein a return path for electrical current is defined through a patient's tissue.

The electrically exposed portion of the guidewire can be located distally and/or proximally with respect to the kink. The electrical circuit can effect monopolar cutting by connecting one end of the guidewire to an electrosurgical generator, wherein the return path of the current passes through the patient. Alternatively, if desired, a guidewire can be configured in a manner such that both the proximal and distal portions of the guidewire are coupled to an electrosurgical generator, and current flows through both portions of the guidewire on both sides of the kink or bend. In this manner, electrically exposed regions can be provided in the guidewire on both sides of the kink, and may be separated by an insulated segment. The electrically exposed regions can “face” each other, wherein electrical current is caused to flow down a first portion of the guidewire, out through a first electrically exposed region on a first side of the kink or bend, across the tissue (acting to ablate the tissue), and back into a second portion of the guidewire through a second electrically exposed region on the other side of the kink or bend. This will permit the current to principally flow through the core wire and not through the patient's body. The surfaces of the facing electrically exposed regions can be provided with elevated peaks, spikes or bumps to further concentrate electrical charge to make current more prone to jump from one electrically exposed region to the other through the tissue. Also, while the treatment of myocardial tissue is discussed herein, the disclosed embodiments can be applied to any suitable tissue structure(s).

The foregoing and other features and advantages of the disclosed technology will become more apparent from the following detailed description of several embodiments which proceeds with reference to the accompanying figures.

### BRIEF DESCRIPTION OF THE FIGURES

FIGS. 1-2 are illustrations of a guidewire in accordance with the present disclosure or aspects thereof.

FIG. 3 is a schematic illustration of aspects of the guidewire of FIG. 1.

FIG. 4 illustrates an embodiment of a kinked denuded guidewire wherein dielectric material, and optionally, radiopaque material, has been removed from the region of the guidewire on both inner sides of the kink. It will be recognized that the guidewire can be provided without insulating material in a desired region by way of a masking process, for example.

FIG. 5 illustrates an embodiment of a kinked denuded guidewire wherein dielectric material, and optionally, radiopaque material, has been removed from the region of the guidewire on one side of the kink but left intact on the other side of the kink. It will be recognized that the guidewire can be provided without insulating material in a desired region by way of a masking process, for example.

FIG. 6 is a schematic illustrating aspects of a procedure for cutting myocardial tissue in accordance with the present disclosure.

FIG. 7A is a further schematic illustrating aspects of a procedure for cutting myocardial tissue in accordance with the present disclosure.

FIG. 7B is yet a further schematic illustrating aspects of a procedure for cutting myocardial tissue in accordance with the present disclosure.

FIG. 8 is an illustration of aspects of a supporting or supporting catheter in accordance with the present disclosure.

FIG. 9 is an illustration of a front end portion of an electrosurgical assembly including a crimped denuded guidewire supported by a pair of supporting catheters in a position prior to cutting.

FIG. 10 is an illustration of a front end portion of an electrosurgical assembly including a crimped denuded guidewire supported by the pair of supporting catheters of FIG. 11 in a position when ready to cut.

FIG. 11 is a depiction of a backend assembly of an electrosurgical system in accordance with the present disclosure showing a proximal end of the supporting or guiding catheters incorporated into the system.

FIG. 12 is an isometric view of a guidewire gripper in an open position.

FIG. 13 is an isometric view of a guidewire gripper in a closed position.

FIGS. 14-15 are cross sectional views of the guidewire gripper of FIGS. 12-13.

FIG. 16 is an isometric view of a guidewire kinker and denuder in accordance with the present disclosure in an open position to receive a guidewire to be denuded and kinked.

FIGS. 17 and 18 are exploded views of the kinker of FIG. 16 showing upper and lower housing portions and a rotatable cutter held in place by the housing portions, and omitting a central lever of the kinker of FIGS. 19-20 for purposes of clarity.

FIGS. 19-20 are isometric views of a central lever of the kinker device of FIG. 18.

FIGS. 21A-21B depict a close up view of a portion of the kinker of FIG. 18.

FIG. 22A is an isometric view of the device of FIG. 16 locked in place to denude and kink a guidewire.

FIG. 22B is a cross sectional view of the device of FIG. 16 wherein the guidewire is about to be denuded with the blade in a starting position.

FIG. 23A demonstrates the direction of rotation of the knob to drag the cutting blade over the guidewire to remove the insulating layer from the guidewire.

FIG. 23B illustrates a cross section of the device as the blade removes the insulating material from the guidewire.

FIGS. 24A-24B illustrate views of the kinker after the cutter has reached its ending position.

FIG. 25A illustrates kinking of the guidewire by folding over the lens portion of the kinker about a pivot axis.

FIG. 25B illustrates a cross sectional view showing the blade in a stowed position to permit kinking of the guidewire placing the blade in a location away from the guidewire.

FIGS. 26A-26G illustrate aspects of a further representative embodiment of a gripper device in accordance with the present disclosure.

FIG. 27 depicts an electrical cable for use with a system as described in the present disclosure.

### DETAILED DESCRIPTION

The present application presents advantages and improvements over systems described in U.S. patent application Ser. No. 16/954,710 (“the '710 application”), filed in the United States of America on Jun. 17, 2020 and U.S. patent application Ser. No. 17/148,616, filed Jan. 14, 2021. Each of these patent applications is incorporated by reference herein in its entirety for all purposes.

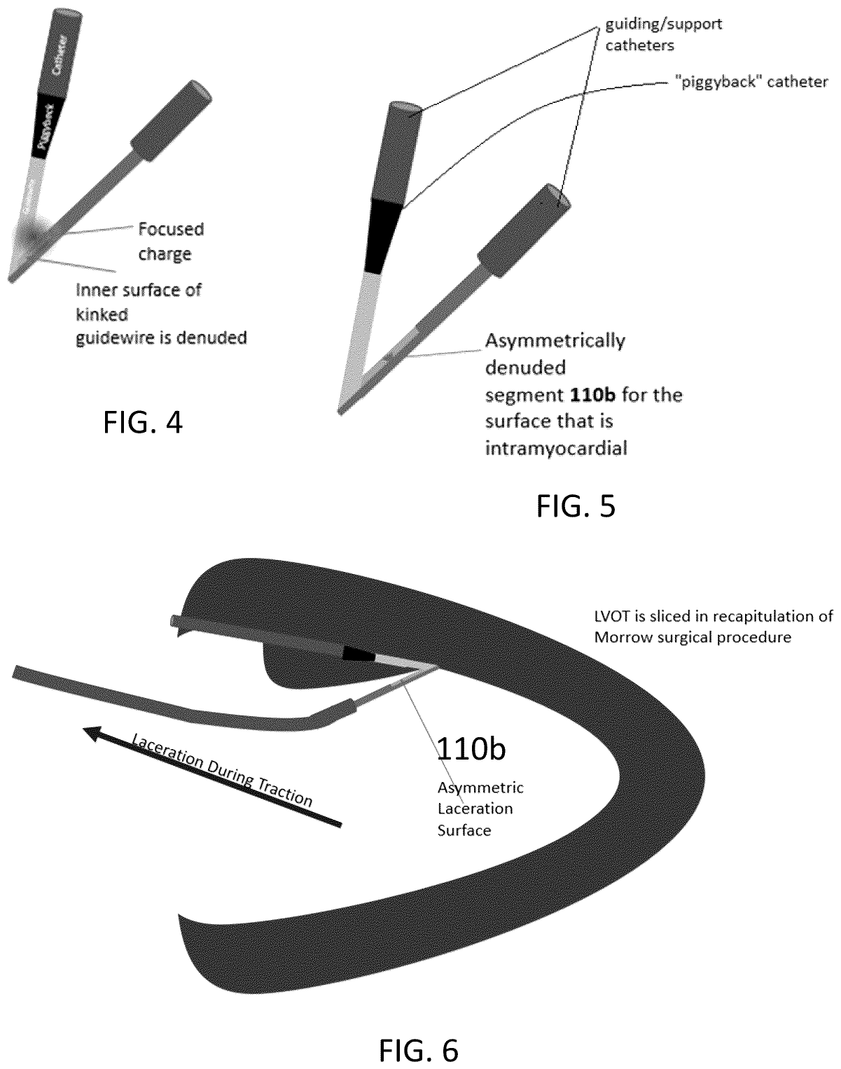

One significant area of improvement in the presently disclosed system as compared to Ser. No. 17/148,616 is a consequence of providing an electrically exposed region of the guidewire on one side of the inner surface of the kinked region of the wire, preferably located distally of the kink. This permits the exposed region to be placed adjacent tissue to be cut, such as by inserting it into and pulling it just beyond a passage defined through a tissue structure to be cut. Stated another way, through a tunnel that has been formed into tissue, wherein the tissue is to be cut along an edge of the tunnel. The tunnel or passage can be defined, for example, by way of electrifying the distal end of the guidewire and burning the passageway through tissue, such as along a desired path through myocardial tissue. In other implementations, other tissue dissection techniques can be used, such as those set forth in International Application No. PCT/US2021/41310, filed Jul. 12, 2021, which is incorporated by reference herein in its entirety for all purposes. The exposed region 110b (see FIG. 4) is placed adjacent tissue to be cut in the passageway, the guidewire is electrified, and tissue proximate the exposed region of the guidewire in the “V” shaped kink is ablated by way of electrical current running through it and traction being applied to the guidewire.

The electrically exposed portion of the guidewire can be located distally and/or proximally with respect to the kink. The electrical circuit can effect monopolar cutting by connecting one end of the guidewire to an electrosurgical generator, wherein the return path of the current passes through the patient. Alternatively, if desired, a guidewire can be configured in a manner such that both the proximal and distal portions of the guidewire are coupled to an electrosurgical generator, and current flows through both portions of the guidewire on both sides of the kink or bend. In this manner, electrically exposed regions can be provided in the guidewire on both sides of the kink, and may be separated by an insulated segment. The electrically exposed regions can “face” each other, wherein electrical current is caused to flow down a first portion of the guidewire, out through a first electrically exposed region on a first side of the kink or bend, across the tissue (acting to ablate the tissue), and back into a second portion of the guidewire through a second electrically exposed region on the other side of the kink or bend. This will permit the current to principally flow through the core wire and not through the patient's body. The surfaces of the facing electrically exposed regions can be provided with elevated peaks, spikes or bumps to further concentrate electrical charge to make current more prone to jump from one electrically exposed region to the other through the tissue. Also, while the treatment of myocardial tissue is discussed herein, the disclosed embodiments can be applied to any suitable tissue structure(s).

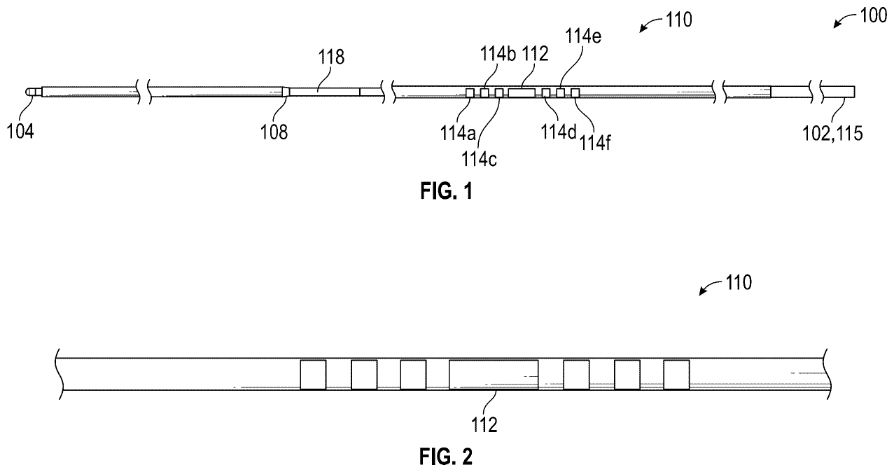

For purposes of illustration, and not limitation, FIGS. 1 and 2 illustrate an example of a guidewire 100 previously described in Ser. No. 17/148,616, having a proximal end 102 and a distal end 104. The guidewire 100 is built upon a core wire 115, such as of stainless steel that is ground down to match a desired profile. As illustrated, the guidewire includes a coating 118 along most of its length formed from an electrically insulating material, discussed in further detail below. As depicted, the proximal and distal ends 102, 104 of the guidewire are exposed to permit them to be coupled to an electrosurgical generator and/or be used to burn through tissue. The core wire 115 can be stainless steel (e.g., Hyten, SLT Type 4) for strength and maintaining straightness.

As illustrated, the guidewire 100 includes a radiopaque marker pattern 110, or pattern of one or more radiopaque markers, disposed over the core wire 115, and underneath the insulating coating 118, to indicate a location along the guidewire 100 proximate a middle section of the guidewire that is to be kinked and used to cut through tissue during an electrosurgical procedure, described in further detail below. The proximal end 102 and distal end 104 of the core wire 115 can be exposed and can be configured to be coupled to an electrosurgical generator. The dielectric coating 118 can be configured to be stripped from the guidewire proximate the radiopaque marker pattern, described in further detail below. The guidewire 100 can have a denuded tip or distal end 102, such as the last 1-5 mm or any increment of 0.1 mm therebetween, and a denuded proximal end 102 (e.g., 0.5-30 mm of length). The proximal denuded region is preferably roughened to enhance physical contact with the spring loaded connector 610 of cable 600. If desired, the marker pattern can be formed from a plurality of marker bands, or a single elongate marker section, or other desired arrangement. To manufacture the guidewire with the markers, in some implementations, an elongate rod can be ground down in the region where the marker(s) are to be located. Next, a radiopaque material, such as gold, platinum, or the like is deposited in the recesses formed by the grinding. Thereafter, the core wire can be plunge ground and shaped to its final diameter prior to coating with dielectric material and/or other coatings. This provides a smooth continuous core wire surface along the length of the core wire including in the region(s) of the marker band(s).

In some implementations, the radiopaque marker pattern 100 can define a central region 112 as described in Ser. No. 17/148,616 that can be crimped and stripped of the dielectric coating 118, and one or more indicia 114(a-f) on either side of the central region. The indicia can be used for purposes of measurement or determining relative distances when conducting a medical procedure. The disclosure of Ser. No. 17/148,616 teaches stripping the guidewire along one side to form an electrically exposed portion that overlaps with and encompasses the kinked region of the guidewire. In accordance with the present disclosure, the electrically exposed region is preferably to one side of or adjacent to the kinked region of the guidewire so that the electrically exposed portion is partially or fully surrounded by myocardial tissue to be cut by ablation techniques using the kinked denuded wire.

In various embodiments, the radiopaque marker pattern 110 can include a radiopaque metallic material. In a preferred embodiment, the radiopaque marker pattern includes gold metallic material deposited over the core wire 115. If desired, the radiopaque marker pattern 110 can include gold metallic material electroplated on the core wire 115 using masking techniques to form the marker pattern 110. Other suitable radiopaque materials can be used, such as platinum and the like.

The radiopaque marker pattern 110 can include an uneven or roughened surface configured to enhance its visual signature under fluoroscopy. The surface roughness can be achieved by various electroplating techniques. If desired, the surface roughness can have a roughness average between about 0.01 micrometers and about 100 micrometers or any increment therebetween of 0.01 micrometers, for example.

In some implementations, and as illustrated in FIG. 1, the radiopaque indicia 110 can include a plurality of spaced marker bands disposed on either side of a central marker band region 112 of the radiopaque marker pattern. For example, the central region of the radiopaque marker pattern 110 can be between about 0.5 cm and 2 cm in length and each of the plurality of spaced marker bands can be between about 0.5 mm and 5 mm in length. In the particular implementation of FIG. 2, the central region of the radiopaque marker pattern can be about 1 cm in length and each of the plurality of spaced marker bands can be about 1 mm in length, wherein each marking can be separated by a gap, for example, of about 1 mm. The longer center marker band (˜1 cm) makes it more visible under fluoroscopy and to the naked eye. It helps a physician identify the mid-shaft of the guidewire 100 (where the marker is preferably located) and indicates the location to be denuded of insulation for the laceration portion of the procedure. The smaller marker bands on each side provide an easily identifiable pattern (e.g., “raccoon tail”) under fluoroscopy and to naked eye. Such a unique pattern reduces confusion with other interventional device markers. Each 1 mm marker band, also allows physicians to measure how far the guide catheter tip is from the laceration surface of the guidewire. It will be appreciated that this marker pattern can simply be a single radiopaque region of the guidewire, or may include a plurality of spaced markers that can be discrete from one another.

In accordance with some further aspects, the dielectric material used to form the insulating layer 118 can have a dielectric strength at 1 mil thickness between about 5600 V/mil and 7500 V/mil. The dielectric material can be any suitable dielectric material, such as a polymeric coating and the like. In some implementations, the dielectric coating is formed in whole or in part from parylene, such as parylene C. The parylene can be deposited over the core wire and the radiopaque marker pattern by way of any suitable technique, such as chemical vapor deposition, for example. The parylene coating is preferably transparent or translucent to permit visual identification of radiopaque marker pattern 110.

In various embodiments, the guidewire can have different dimensions and thicknesses. In some embodiments, the guidewire has an outside diameter of about 0.014 inches, including the thickness of radiopaque markers and coatings. The dielectric material coating can have a thickness between about 0.1 mil and about 20 mil, for example, or any increment therebetween of about 0.1 mil.

In various implementations, the core wire can include at least one section of reduced diameter in the region of the radiopaque marker pattern. For example, the region of the core wire 115 in the region of the radiopaque marker pattern 110 can be ground down to provide an elongate recessed region to accommodate the radiopaque marker pattern(s). This can be done to maintain the profile of the guidewire along its length and to ensure that its finished thickness including any coatings does not exceed 0.014 inches. The radiopaque marker pattern can have a thickness, for example, between about 0.0005 inches and about 0.0010 inches, or any increment therebetween of 0.0001 inches.

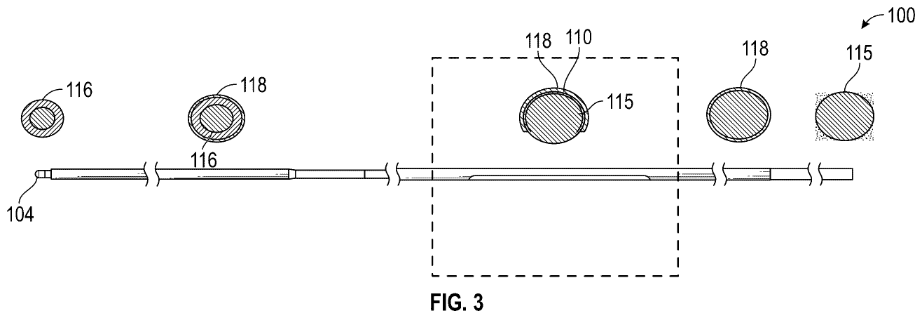

With reference to FIG. 3, if desired, a radiopaque coil 116 can surround the distal tip 104 of the guidewire 100 that is made from platinum or other suitable (and preferably radiopaque) material, such as a mixture of 90/10 platinum/iridium. FIG. 3 illustrates the relative placement of overlapping layers of material along the length of guidewire 100. With reference to the left, or distal, end of the guidewire 104, the core wire 115 is surrounded by coil 116. The spring coil can be laser welded to the core wire 115, for example, to withstand RF energy delivery therethrough. Immediately proximal to the distal end, layer 118 of insulating material begins, and extends to the proximal end of the guidewire at right. The central region of the guidewire is illustrated as having already been denuded of layer 118 and part of layer 110 using a denuder, as discussed below, to permit an electrosurgical procedure as set forth herein below to be carried out.

When denuding this region of the guidewire, typically about 3-6 mm of the guidewire is denuded, but this can be larger or smaller, in increments of 0.5 mm, as desired. A small controlled denuded region allows for better concentration of energy to lacerate tissue and provides a steady cutting discharge. When denuded and kinked, the parylene coating and the radiopaque marker band underneath are scraped to reveal a “laceration” surface for which to delivery RF energy therethrough. Only the parylene coating needs to be denuded to deliver energy, but denuding the gold-plated marker band allows for visual confirmation of denudation because the coating can be clear, if so configured.

FIG. 5 illustrates the guidewire 100 after it has been denuded along a region 110b to expose the core wire and after it has been kinked, illustrating the relative placement of section 110b to the location of the kink in the guidewire. As illustrated, the guidewire has been bent past 90 degrees to an acute angle. The location of the denuded section can be contrasted with that in FIG. 4, which places the electrically exposed area across the kink and on both sides of the kink. By placing the electrically exposed region on only one side of the kink, less tissue is exposed to electrical current, permitting more targeted application of energy in the disclosed procedures.

For example, FIG. 6 illustrates modification of a left ventricular outflow tract obstruction (LVOT) from myocardial hypertrophy or hypertrophic cardiomyopathy. The tissue is presented in cross section, wherein a passageway has been formed through the tissue along the ventricular wall by ablation or other suitable technique as set forth above. The distal end of the guidewire is captured after passing through the distal end of the passage (right end of passage in the figure) and the guidewire is advanced until the apex of the kink of the guidewire is straddling tissue at the end of the passageway as indicated in the figure. Further, it can be appreciated that the electrically exposed region 110b of the guidewire is located distally on the guidewire with respect to the location of the kink in the guidewire so that the electrically exposed region can physically contact the tissue to be cut by the application of electrical current when the guidewire is in the position indicated in FIG. 6. Traction is applied to both ends of the guidewire while the guidewire is energized and the guidewire is advanced proximally is the tissue is cut, until the passageway has been fully traversed, resulting in the tissue being lacerated along the predetermined path defined by the passageway. This procedure can be conducted one or more times to form one or more lengthwise lacerations to splay and enlarge the LVOT (referred to herein as the SESAME procedure), wherein the enlargement will increase over time.

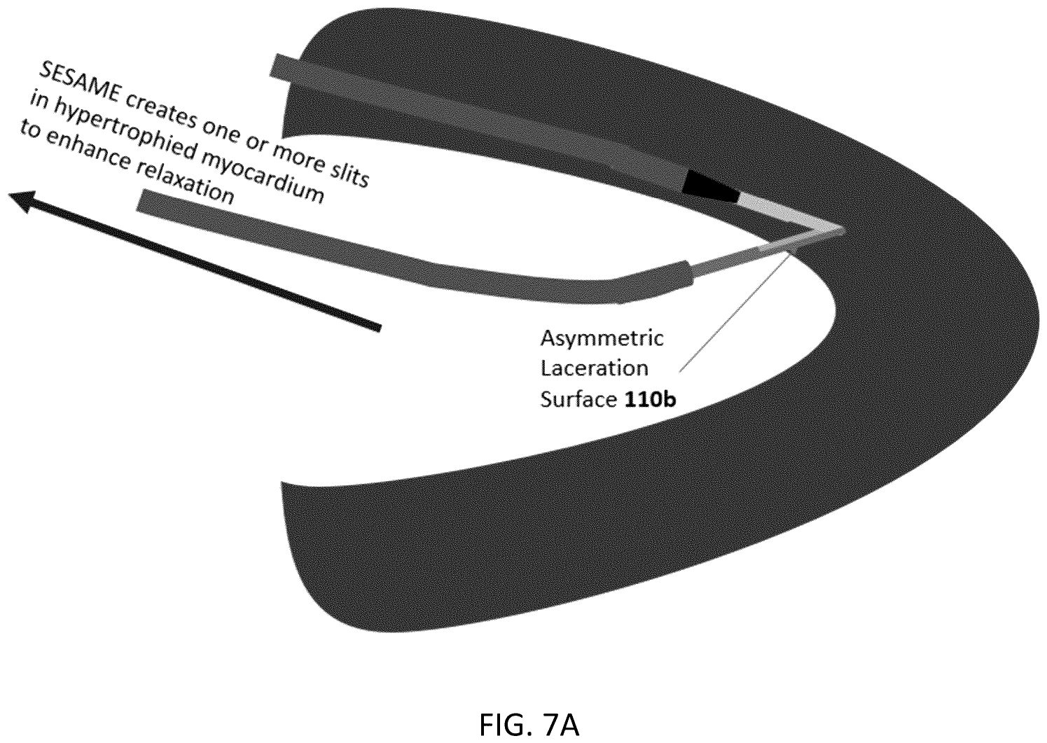

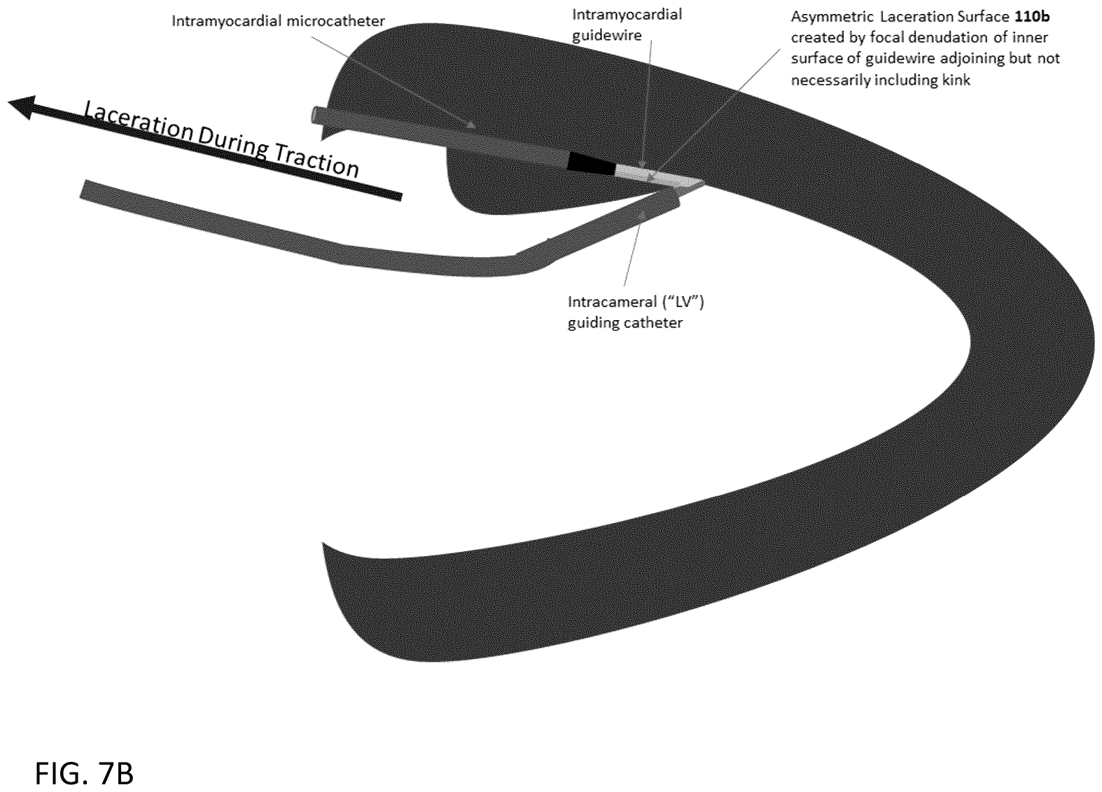

FIG. 7A illustrates a further cross section illustrating a further procedure to be performed on a left ventricular wall of a patient using the disclosed electrosurgical system to treat left ventricular diastolic dysfunction (heart failure with preserved ejection fraction, HFpEF) with impaired relaxation. Heart failure with preserved ejection fraction (HFpEF) occurs when the lower left chamber of the heart (left ventricle) is not able to fill properly with blood during the diastolic (filling) phase. The amount of blood pumped out to the body is then less than normal, and less than what the body needs. Applicant has appreciated that this can be a result of the ventricular wall being too stiff to relax, prohibiting the ventricle to fill with blood. In accordance with the disclosure, the disclosed electrosurgical system can be used to form one or more lengthwise lacerations to splay and enlarge the left ventricular chamber, increasing both end-diastolic and end-systolic dimensions, to improve diastolic relaxation from “concentric” hypertrophic or infiltrative cardiomyopathies. This can be accomplished by forming a passageway along and below the inner surface of the left ventricle using a tunneling technique as mentioned above using an electrosurgical guidewire and/or other techniques. Then a procedure as described above with respect to FIG. 6 can be performed by advancing the distal end of the electrosurgical guidewire through the distal end of the passageway defined through the wall of the left ventricle, capturing the distal end of the guidewire and externalizing it while further advancing the guidewire through the passageway until the kink of the electrosurgical guidewire has emerged from the distal end of the passageway, and the electrically exposed region 110b is facing at least partially in a proximal direction, so that when the guidewire is electrified, electrical current flows through the electrically exposed region 110b into the tissue to be cut, and pulling both ends of the guidewire proximally will effectuate the cut along the entire length of the passageway defined along the ventricular wall, resulting in a laceration and splaying of myocardial tissue, the location of which is defined by the location of the passageway. This process can be repeated any desired number of times until sufficient flexibility has been imparted to the ventricular wall to permit it to relax and expand to accommodate more blood while it is filling.

FIG. 7B illustrates a further implementation of a SESAME procedure for treating hypertrophic cardiomyopathy. This implementation of the device uses a (e.g., 4 mm) inner surface denudation of wire insulation between the kink in the wire and an electrically insulating intramyocardial microcatheter. An asymmetric laceration surface 110b that is electrically exposed is created by focal denudation of the inner surface of the guidewire (i.e., on the “inner” surface of the kinked region) that can adjoin, but may or may not include denuding the insulation in the kink itself. So, the electrically exposed region 110 is within the tunnel that is defined earlier during the procedure permitting ablating current to flow directly into tissue that is adjacent the electrically exposed region, resulting in ablation of that tissue. A contralateral intracameral guiding catheter is situated in the left ventricle (LV) and rides along the endocardial surface of heart during the cutting operation, almost touching the kinked region. A microcatheter can also be disposed within the guiding catheter to enhance electrical insulation of the overall system.

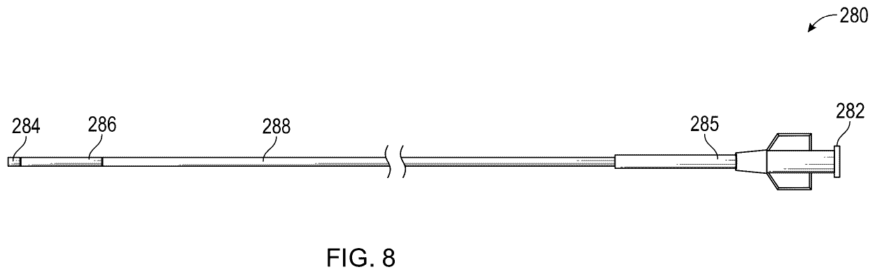

The disclosure further provides implementations of an electrosurgical system. For purposes of illustration, and not limitation, FIGS. 8-11 illustrate aspects of such a system, including an electrosurgical generator 800 coupled to proximal end 102 of guidewire 100. The system further includes a pair of supporting catheters 280. This presentation is illustrative and while the figures are principally directed to procedures set forth in Ser. No. 17/148,616, it will be appreciated that slight modification of the distal ends of the support catheters 280 can be made to perform the SESAME and related procedures.

With reference to FIG. 8, as illustrated, each guiding or supporting catheter 280 has a proximal end 282 and a distal end 284 and defines an elongate lumen along its length. The guiding catheter can also have the beneficial effect of enhancing electrical insulation already present on the guidewire 100 by effectively surrounding the guidewire with even more insulation. However, use of the guiding catheter(s) 280, or any intermediate catheter passing over the guidewire 100 but within the catheter can be practiced to enable guide-wire exchange if needed and to protect the myocardium against “cheese-cutting” effect when traction is applied by spreading the stress over a larger surface area of tissue.

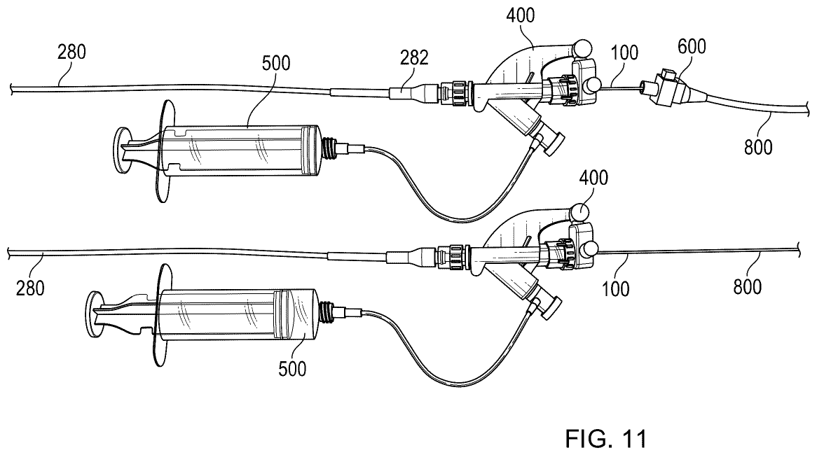

The proximal end 282 of the catheter includes a connector having a flange to couple to the proximal end 410 of gripper 400 as set forth in FIG. 11. The main body of the catheter 288 is formed by an elongate polymeric tube made, for example, from a high viscosity polyamide material, such as Vestamid MLL21 (polyamide 12), having a Vicat softening temperature of 140 C according to ISO 306 and a Shore hardness of about 75D, discussed in further detail below, and a length, L3, between 30 and 50 cm, for example. The main body 288 transitions to an intermediate region 286 near the distal end formed preferably from a material with a somewhat lower durometer, such as a polyether block amide (e.g., Pebax® elastomer) having a shore hardness form about 60D to about 72D and a relatively short length L2 of between 1 and 5 cm or any increment therebetween of 1 mm, such as a length of 2 cm. A distal tip portion of the catheter at location 284 can be made from a still softer material, such as Pebax material having a softness between 35D and about 45D, such as about 40D. The distal tip portion can be short, having a length, L1, such as between 2 and 5 mm in length, such as 3 mm. Catheter 280 further can include a strain relief section 285 between, for example, 0.5 and 15 cm in length, to provide a transition in stiffness between the connector at the proximal end 282 of the catheter and the main body of the catheter 288. Sections 286, 288 can have a diameter, for example, between 0.08 and 0.09 inches, or any increment therebetween of 0.001 inches. The tip region 284 can have a diameter, for example, between 0.07 and 0.08 inches, or any increment therebetween of 0.001 inches. This basic configuration of catheter 280 can be shaped as desired depending on the anatomy being worked on in the procedure. The shape of the distal portions of each supporting catheter can resemble the shapes set forth in FIGS. 6-7, or any other desired shape to suit the anatomy.

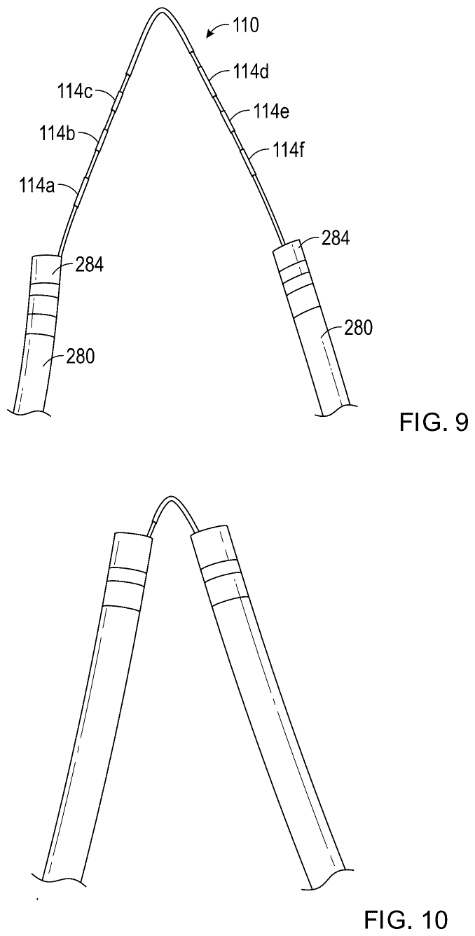

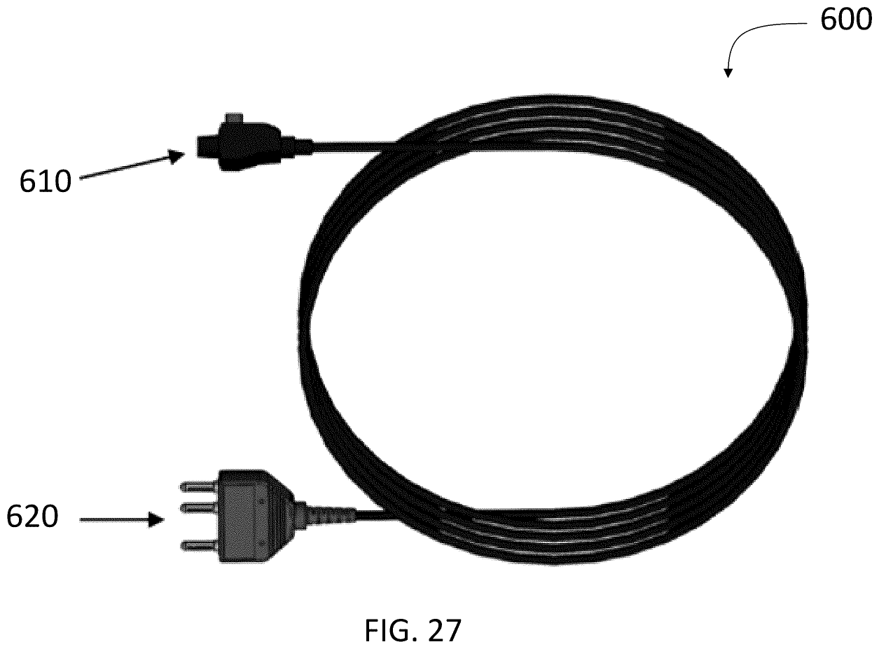

The system further includes a guidewire 100 as set forth herein that is kinked (FIGS. 9, 10) and electrically exposed in the central region of the radiopaque marker pattern 110. The guidewire 100 is placed with the kink therein straddling the end of a passageway defined through myocardial tissue using any of the supporting catheters set forth herein, as appropriate. The position of each of the supporting catheters is adjusted with respect to the guidewire 100 between the kinked central region 112 of the radiopaque marker pattern 110 and the proximal 102 and distal 104 ends of the guidewire. The proximal end of the core wire 115 is coupled to the electrosurgical generator 800 at least in part by way of a respective connector cable 600, shown in further detail in FIG. 27. Connector cable 600 includes a first end 610 with a spring loaded connector to connect to the proximal end 102 of the guidewire 100, and a second end having a plug 620 to connect to an electrosurgical generator, such as the Medtronic Force Triad and FT-10.

As set forth in FIGS. 9 and 10, distal end 284 of each said supporting catheter 280 is spaced from the kinked portion of the guide wire 100 by aligning the distal end 284 of each supporting catheter with measurement indicia 114a-f disposed on either side of the kinked portion of the wire 100 to space the distal end of each supporting catheter from the electrically exposed portion of the core wire in order to prevent the distal ends 284 of the supporting catheters 280 from being damaged by current flowing across the electrically exposed portion of the core wire 115.

As alluded to in FIGS. 5, 6, and 7, a second proximal supporting catheter, such as a “piggyback” catheter can be used to provide additional columnar support to the guidewire 100 as the guidewire is advanced through the myocardium. The larger support catheter 280 can be introduced over this piggyback catheter to provide still additional support. The piggyback catheter can be removed prior to installation of the grippers to provide an annular passage defined between the outer surface of the guidewire and the inner surface of the support catheter 280 to provide a passageway to direct flushing fluid, such as dextrose solution, to the site as described elsewhere herein.

As mentioned above, the lumen of the guide catheters 280 are preferably large enough to deliver a flush of dextrose to prevent charring during energy delivery. Specifically, during the cutting operations, a dextrose solution is flushed through the catheters from reservoir 500 by way of conduits connecting to ports 440 in grippers 400. The flush can be manual through the catheters 280. The flow rate of dextrose solution can be, for example, 5-10 cc per second, during tissue laceration using the denuded section of guidewire.

If desired, and with continuing reference to FIG. 11, implementations of the electrosurgical system can further include a gripper 400 coupled to a proximal end of each supporting catheter 280.

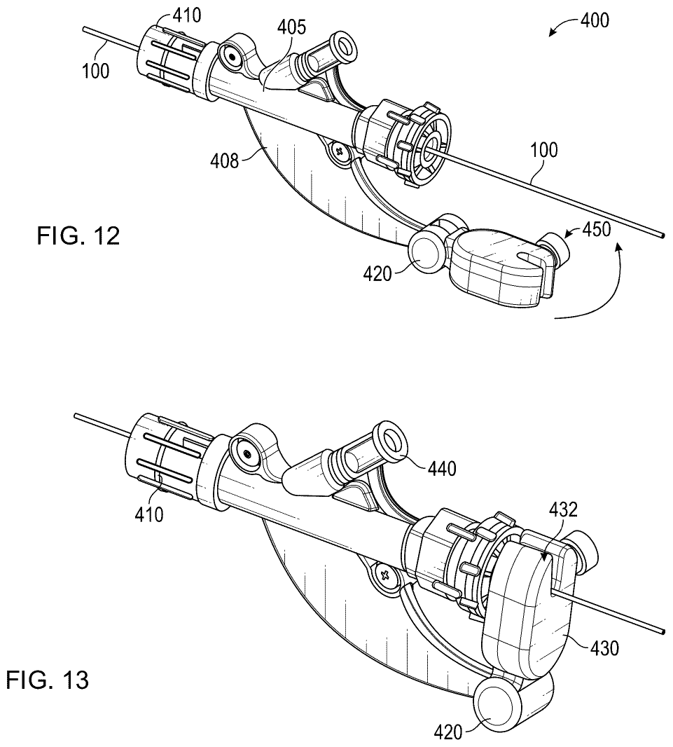

For purposes of illustration, and not limitation, with reference to FIGS. 12-15, each gripper 400 can be configured to be selectively coupled to the guidewire 100 using a lock at the proximal end of the gripper 400, and to the supporting catheter 280 at its distal end 410 to permit the relative position of the guidewire 100 and the distal ends 284 of the catheters 280 to be fixed. This helps to prevent the distal ends 284 of the supporting catheters from being melted.

As depicted in FIGS. 12-13, each gripper 400 is characterized by a main body 405 that defines a lumen therethrough (not shown) to permit passage of the guidewire 100. The main body includes a lock 410 at the distal end to engage a guide catheter, and a proximal end through which an end of the guidewire 100 extends. A port 440 such as for flushing fluid (e.g., dextrose solution) through the guide catheter is disposed on an upper side of the main body in a y-connector arrangement, and defines a lumen therethrough into the lumen defined along the length of the main body. A lower arm or wing 408 extends downwardly from the main body of the gripper 400 and terminates in a hinge 420, to which a rotatable arm 430 is mounted that in turn includes a clamp 450. The arm 430 is rotated about the hinge 420 and defines a groove 432 therethrough to receive the guidewire 100, where the guidewire is clamped in place, as discussed below.

To use the gripper, the wire is inserted through the lumen of the body 405 of the device. The arm 430 of the gripper is flipped up about hinge 420. The wire 100 falls into the channel 432 defined in the arm 430. The screw 450 is then tightened to advance the grip plate 452, which may also include a roughened surface that faces the wire 100, to hold the wire 100 in place. Grip plate 452 is adjacent a block 451, which may be formed from metal or plastic and contained within and held in place by housing or shell 455. Providing some vertical distance between screw 450 and screws 454 provides a sufficient distance for the plate 452 to bend about the point defined by screws 454 when screw 450 is tightened.

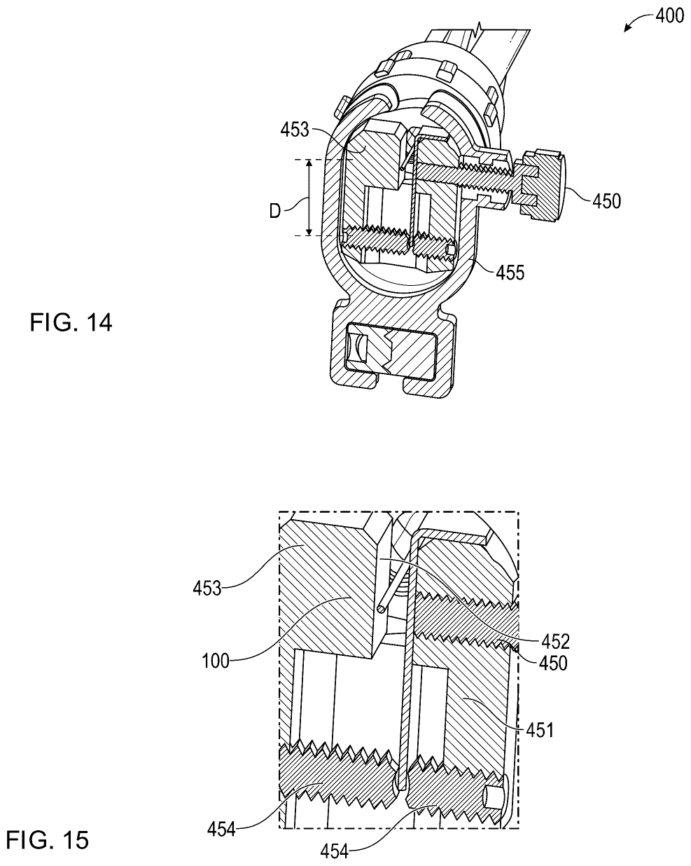

With reference to FIGS. 14-15, a cross section of the arm 430 of the gripper 400 is illustrated. The grip plate 452, made from plastic or metal (e.g., NiTi alloy) is fitted into the groove defined through the arm 430. The clamp 450 includes a rotatable screw threadably received within block 451 that, when rotated, pushes the plate 452 into the groove and pins the guidewire 100 against an opposing wall formed by block 453 that defines the groove. Screws 454 can be used to hold plate 452 in place. The movement of grip plate 452 as illustrated then is a cantilevered movement into the gap due to advancement of screw 450, and bending occurs about the clamped connection formed by screws 454.

The disclosure further provides a kinker to kink and denude the core wire in the central region of the radiopaque marker pattern.

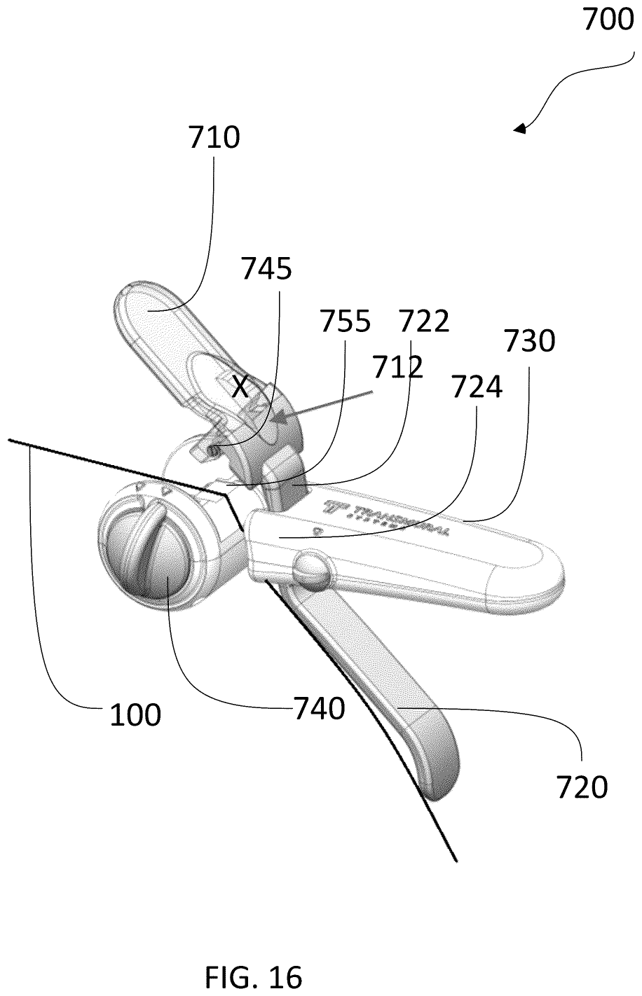

For purposes of illustration, and not limitation, FIGS. 16-24 illustrate aspects of still a further representative implementation of a kinker device 700 in accordance with the disclosure that is configured to controllably denude a portion of the core wire 115 of the guidewire 100. The kinker 700 can include a first arm, such as a first handle 730 and a second arm 720, such as a second handle, joined at a rotatable hinge including an axle 748 and a corresponding journal. The kinker 700 can be configured to hold the electrosurgical guidewire 100 in place with respect to the first arm 730 and second arm 720 to permit the electrosurgical guidewire 100 to be kinked when the first arm and second arm are folded at the rotatable hinge.

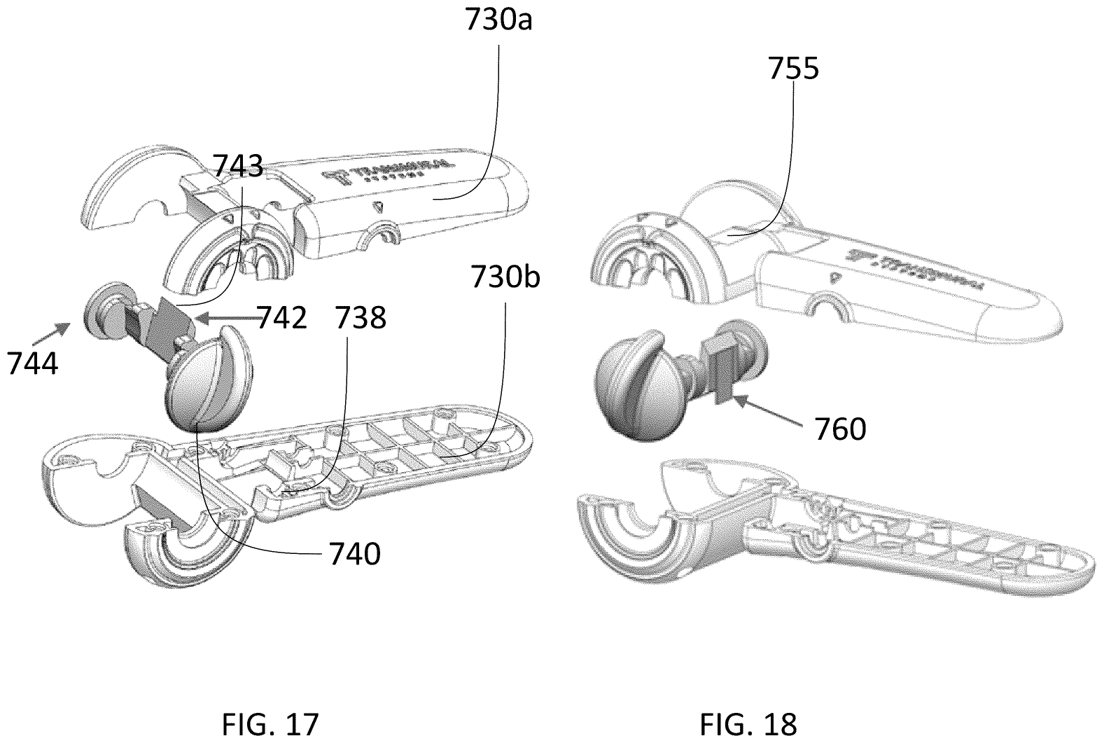

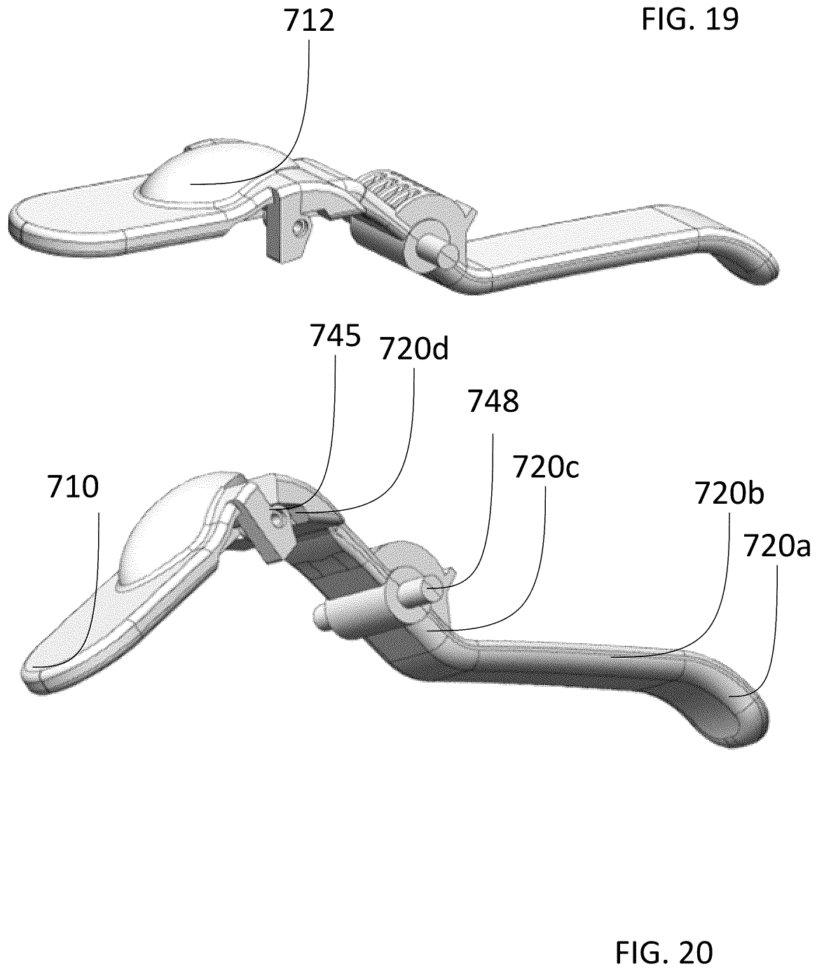

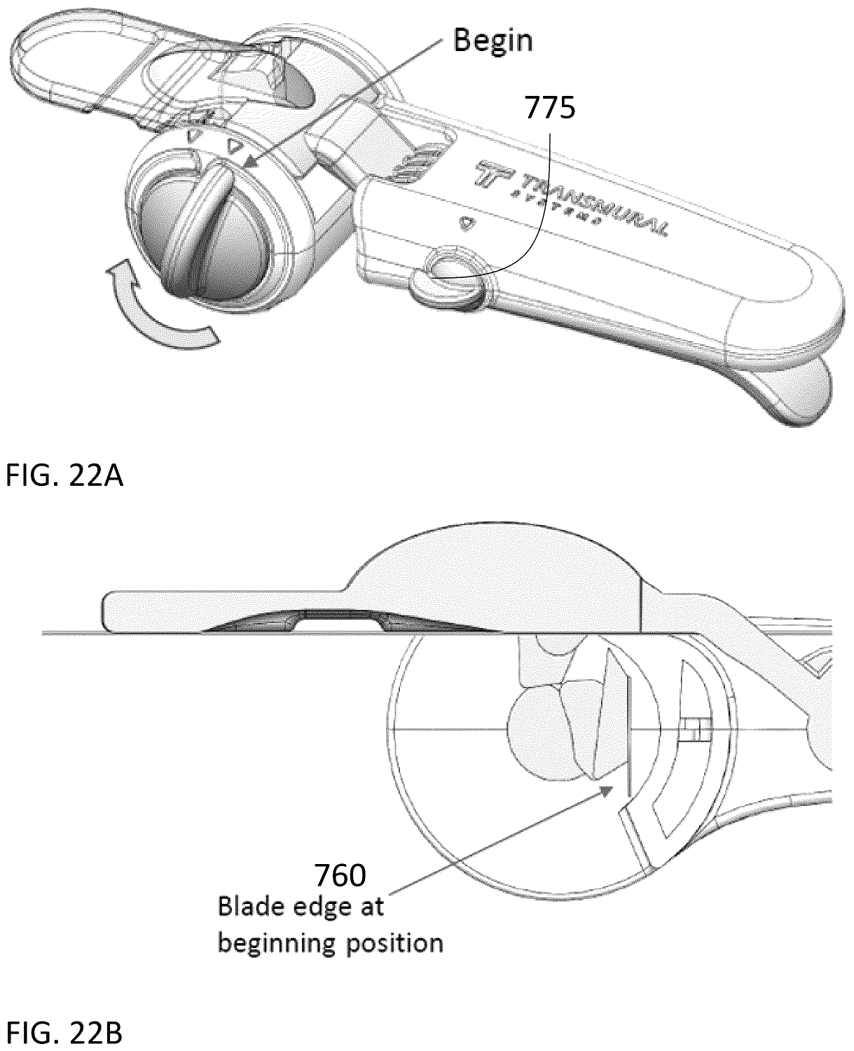

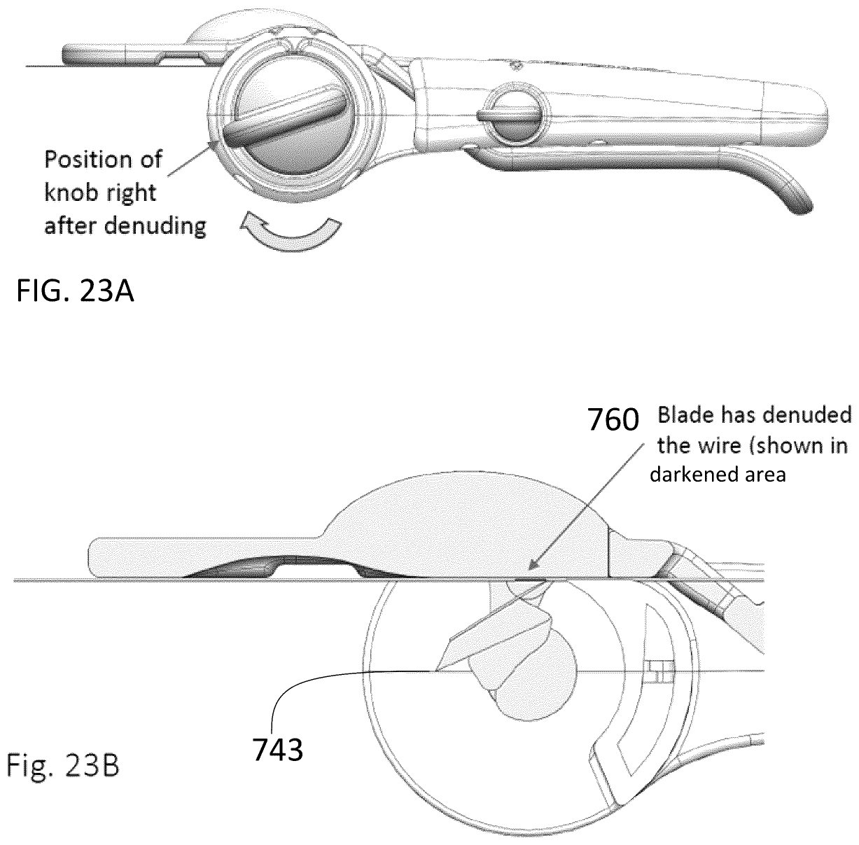

With reference to FIGS. 16-20, the kinker 700 includes an elongate handle 730 that forms a main body of the device. A distal end of the handle 730 terminates in a rotatable joint that houses a rotating cutter 740. As shown in FIGS. 17-18, the handle 730 is formed from an upper portion 730a and a lower portion 730b, which cooperate to surround bearings formed into the cutter 740 such that the cutter 740 is rotatably disposed in the handle between a beginning position illustrated in FIG. 22A, and an end position illustrated in FIGS. 23A, 24A and 25A. The kinker 700 also includes a lever assembly 720 (FIGS. 19-20) that is rotatably received between the housing portions 730a, 730b. In particular, a fulcrum, or bearing 748 is rotatably received in a journal 738 formed into the housing portions 730a, 730b. Lever assembly 720 includes a first handle portion that is pulled against the housing 730 when it is squeezed by a user. The handle portion begins at a proximal rounded end 720a that bends into a straight section 720b along a distal direction. The straight section 720b then bends to form an inclined, straight section 720c that includes bearing 748, which includes two axle ends. Section 720c then extends to a distal end 720d that includes a hinge point 745.

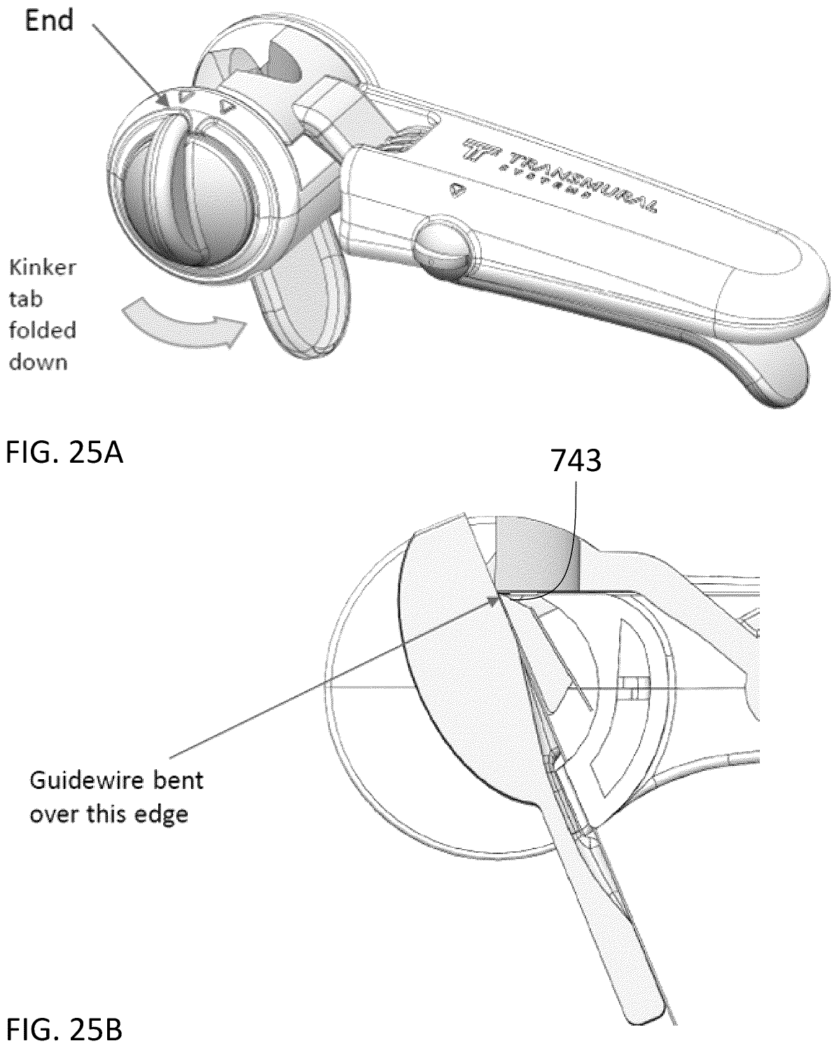

Flap or tab 710, including a window 712 that may have a lens element, is hingedly connected at hinge point 745. As discussed below, after the wire 100 is denuded, the flap or tab 710 is folded over to kink the guidewire within the kinker 700. As illustrated in FIG. 18, window 712 may be included with indicia X for aligning a guidewire 100 with platform 755 defined on the main body 730 of the device 700. Indicia X can also be provided, such as in the form of alignment markings or lines, on surface 755. Window 712, as depicted, includes a magnifying lens to indicate when the guidewire 100 has been properly aligned with the kinker 700. With reference to FIG. 16-17, the cutter 740 includes two ends having arcuate bearing surfaces that are rotatably supported by corresponding journals formed into the main body 730. A central portion of the cutter includes a plate portion 742 that has a cutting blade 760 mounted thereon and a posterior edge 743 that the guidewire 100 is bent over (FIG. 25).

The tab 710 can be made, for example, from polycarbonate with a curved surface in the lens region 712 to visually magnify the wire. Markers can be provided on the bottom surface of the tab 710 to help a physician to align the guidewire.

The cutting blade 760 has an exposed cutting edge that sweeps out an arcuate path as the cutter 740 is rotated, wherein the clearance between the cutting edge and the guidewire is such that the cutting edge scrapes off material as the cutter handle is rotated.

As illustrated in FIGS. 22A-B, at a starting position, the cutting blade is protected by a portion of the housing and cannot contact the guidewire. At this point, a physician aligns the gold markers to indicia or marks on the top tab, for example. The handle 720 can then be squeezed against handle 730 to engage the spring lock 775 in the handle 730.

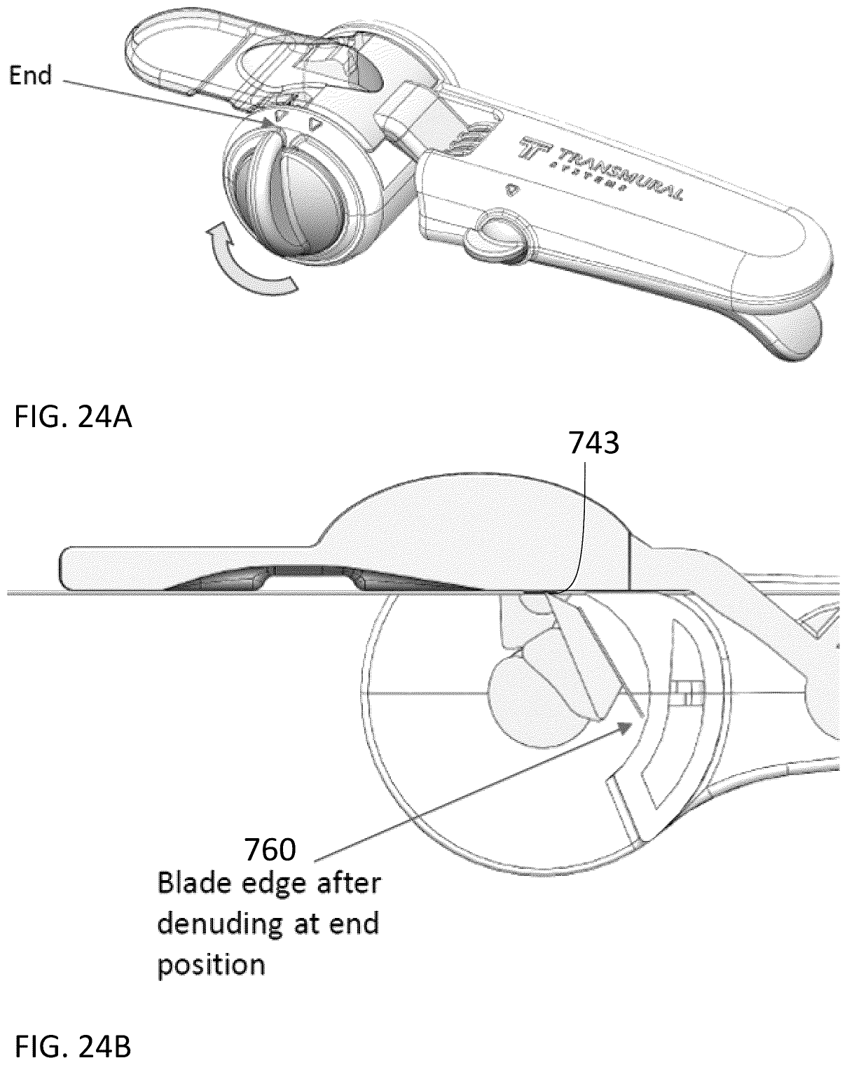

FIGS. 22B, 23B, 24B and 25B illustrate cross sections of the device in the same orientation of the device as presented in FIGS. 22A, 23A, 24A and 25A, respectively. FIGS. 23A-23B show the cutter 740 rotated through a portion of its range of motion until it has just scraped the guidewire 100. During the motion, up to this point, the blade 760 scrapes across the guidewire 100. FIGS. 24A-24B show the blade in the end position where it once again is safely guarded from the guidewire.

With reference to FIGS. 25A-25B, when the knob is fully turned to the end position, the back of the blade 760, edge 743, is positioned such that the wire can be kinked over edge 743. In contrast to the kinker set forth in Ser. No. 17/148,616, the edge 743 can be radiused rather than being a sharp edge to provide more of a radius of curvature to the kink in the guidewire.

Next, tab 710 can be folded down about hinge 745, and the guidewire 100 is bent or kinked at the correct angle, after denuding is complete, to permit the electrosurgical procedure to be performed. The handle 720/730 can be squeezed a second time to release the spring lock 775 and open the handle to permit the kinked guidewire 100 to be removed.

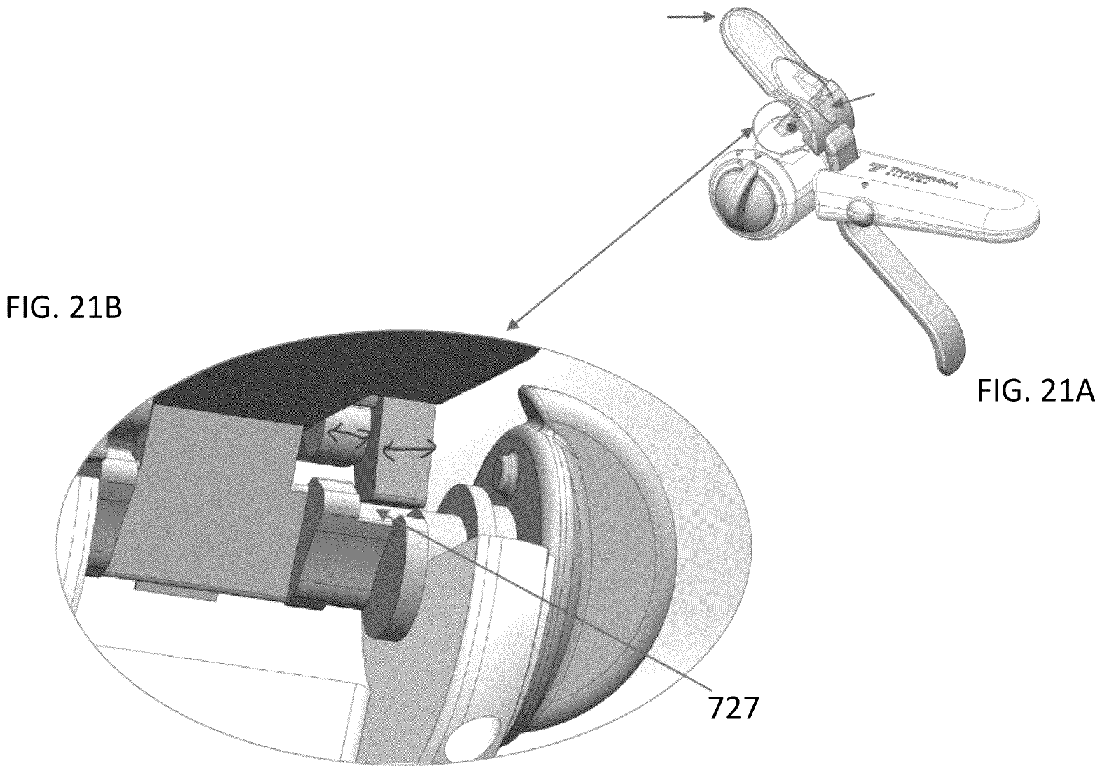

FIG. 21A-B shows an enlarged view underneath the kinker 700 when it is in an open position to receive the guidewire 100. As can be seen, a notch 727 is defined in the axle of the rotating cutter 740 to permit the flap 710 of the handle assembly 720 to fold down when the cutter blade 760 is in the stowed final position as set forth in FIG. 24B, and prevents the flap or tab 710 from being folded before the cutter 740 is in the final, or end position. This is a kinker lock out mechanism that prevents kinking of the guidewire 100 prior to denuding. With reference to FIG. 22A, a spring loaded lock having an actuator 775 is received in the handle to hold the handle 720 in place against the handle 730, and to hold the guidewire in place between a lower surface of the flap and the surface 755 of the main body or handle 730.

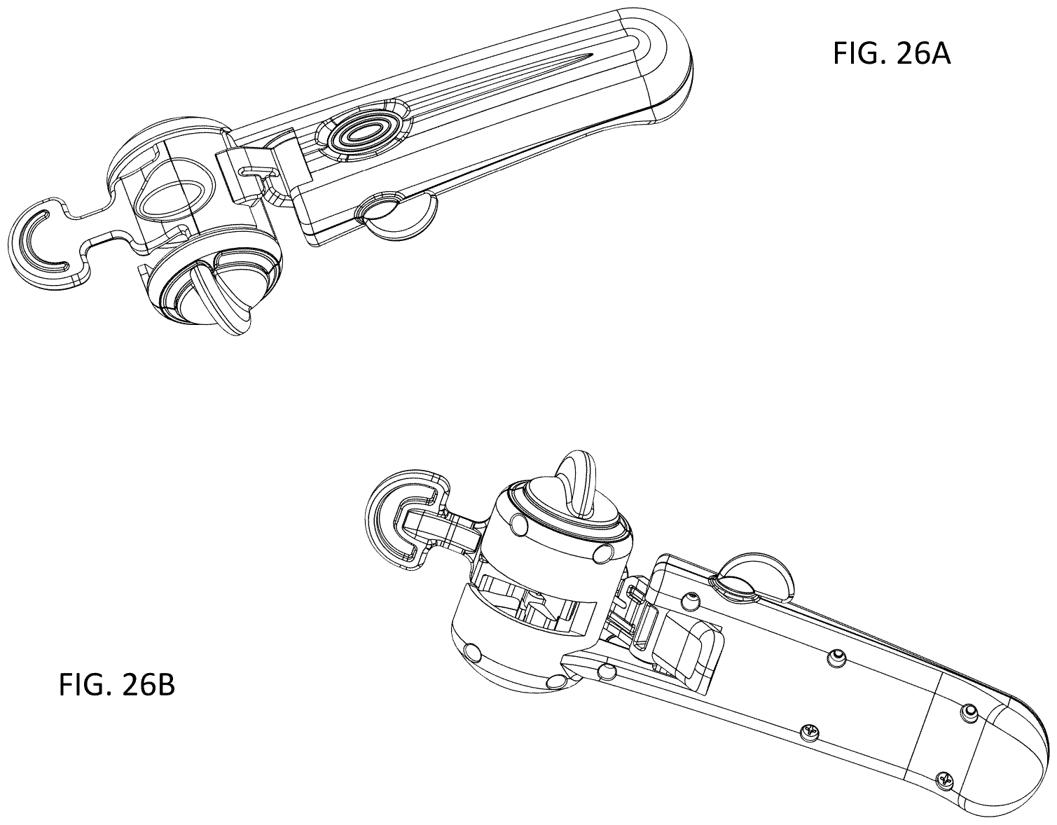

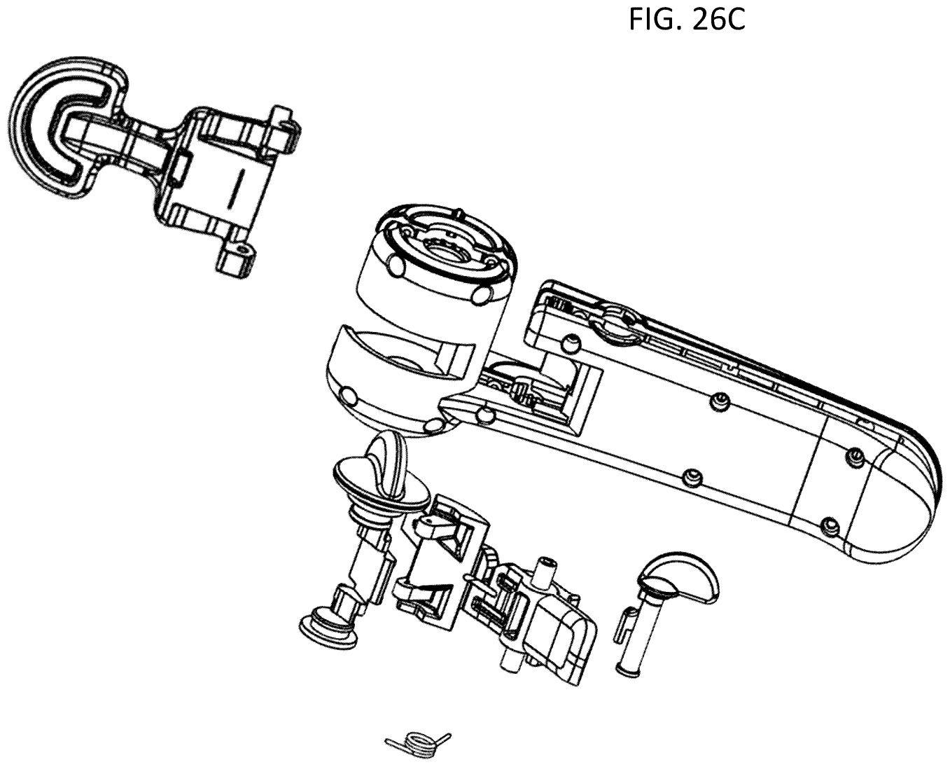

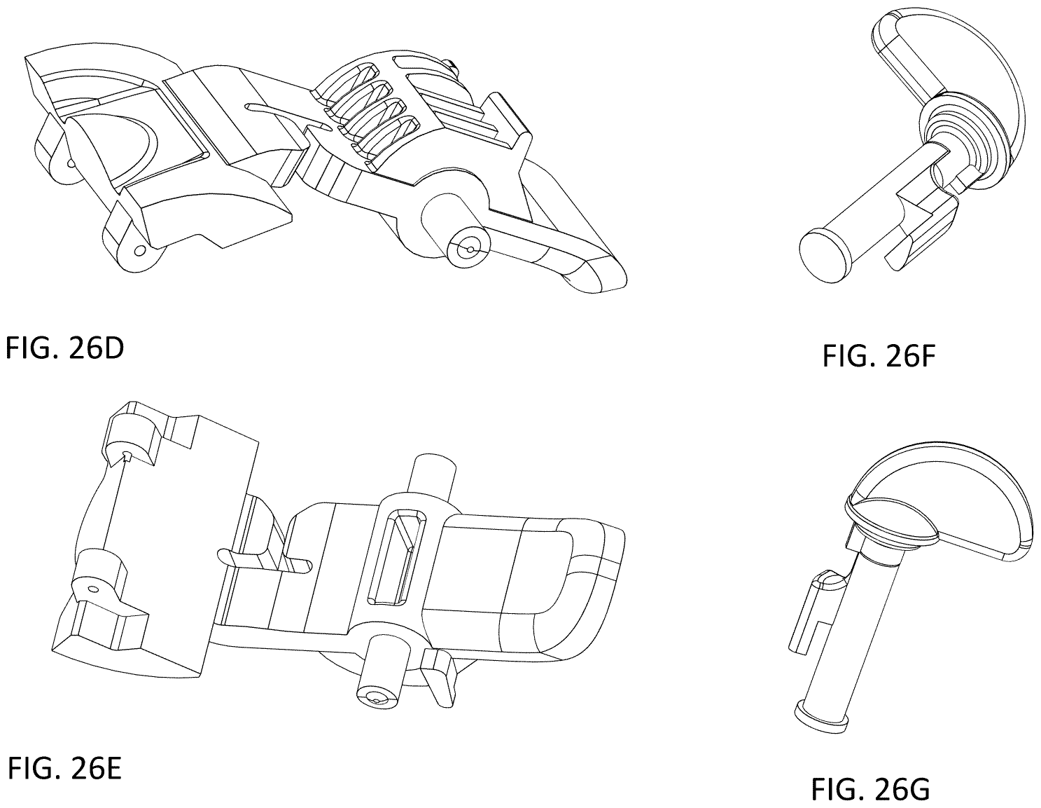

FIGS. 26A-26G illustrate aspects of a further representative embodiment of a gripper device in accordance with the present disclosure. This implementation of the kinker/denuder operates in the same manner and has substantially the same components as the previous embodiment, with several differences. As with the prior embodiment, the kinker includes an elongate handle that forms a main body of the device. A distal end of the handle terminates in a rotatable joint that houses a rotating cutter as with the previous embodiment. The handle is similarly formed from an upper portion and a lower portion, which cooperate to surround bearings formed into the cutter such that the cutter is rotatably disposed in the handle between a beginning position similar to that illustrated in FIG. 22A, and an end position similar to that illustrated in FIGS. 23A, 24A and 25A. The kinker also includes a lever assembly that is rotatably received between the housing portions. In particular, a fulcrum, or bearing is rotatably received in a journal formed into the housing portions. The lever assembly includes a first handle portion that is pulled against the housing when it is squeezed by a user. The lever of this further embodiment is shorter than the prior embodiment to help facilitate one hand operation of the device. A detailed view of the lever assembly can be seen in FIGS. 26 C, 26D, and 26E. A target location for a user's thumb, in the form of an oval shaped grip, is provided to help facilitate proper digit alignment. The handle similarly ends in a hinge point to pivotally receive a respective flap or tab 710, including a window that may have a lens element. After the wire is denuded, the flap or tab is folded over to kink the guidewire within the kinker. The window may be included with indicia for aligning a guidewire with the platform defined on the main body of the device.

With reference to FIGS. 26A, 26B, 26F, and 26G, a spring loaded lock having an actuator, similar to actuator 775 in the previous embodiment, is received in the handle to hold the handles in place and to hold the guidewire in place between a lower surface of the flap and the surface of the main body or handle. The handles can be squeezed together to lock the mechanism, and the lever of the actuator can be flicked with a user's finger of their hand that is gripping the device to cause the spring loaded lock to release to cause the arm and handle to pivot and separate, thereby permitting the kinked, denuded guidewire to be removed from the device.

In further accordance with the disclosure, a kit is provided to perform an electrosurgical procedure, including a guidewire as set forth herein, catheters as set forth herein, and, grippers as set forth herein, and a kinker and denuder to kink and denude the core wire in the central region of the radiopaque marker pattern. It will be appreciated that any of the illustrated embodiments can be used to form such a kit.

An illustrative method includes coupling a proximal end (e.g., 102) of an electrosurgical guidewire as set forth herein to an electrosurgical generator, directing a distal end of the electrosurgical guidewire (e.g., 104) into the patient's vasculature through a catheter to a section of tissue to be tunneled through, such as a section of myocardium, energizing the electrosurgical generator (e.g., 800) to energize the distal exposed end of the electrosurgical guidewire, and burning the myocardium to form a passageway therethrough.

The method can further include advancing the electrosurgical guidewire through the myocardium, and capturing the distal end of the electrosurgical guidewire with a snare catheter. Suitably configured guiding catheters can be used to direct and/or capture the guidewire with a snare catheter. This can be accomplished, in some implementations, by using a deflectable guiding sheath to direct a “hockey-stick” guiding catheter retrograde across the aortic valve to engage the base of the left ventricular septal surface and provide counter-force to enter the septum with a guidewire, mechanically. * * *

After the passageway is formed through the myocardium, the guidewire 100, which is typically about 300 cm in length, is directed into the patient through a first supporting catheter, and out through a second supporting catheter, and passes through the passageway defined through the myocardium

At this point, the radiopaque marker region 110 of the guidewire is still outside the patient and has not yet been introduced. The guidewire can then be kinked and denuded using the kinker 700, while outside of the patient, taking care to denude the guidewire at the inside of the kink at a location distal to the kink. The kinked portion of the guidewire 100 can then be advanced into the patient's anatomy until the kinked portion of the guidewire has emerged through the distal opening of the passage defined through the myocardium. At this point, the grippers 400 may be attached to the catheter proximal ends to build the system of FIG. 11, which can also include reservoirs or syringes 500 including dextrose solution coupled to the Y-connector 440 of each respective gripper to inject fluid through the catheters as needed to the tissue cutting site.

With reference to FIGS. 9 and 10, the denuded kinked portion 110b/110c of the guidewire, which now straddles the passageway defined through the myocardium (not shown) can be used to form an electrosurgical instrument in cooperation with the catheters. This can be done under fluoroscopy by viewing the radiopaque (e.g, gold) marker band(s), as well as one or more radiopaque markers located at the distal ends 284 of the catheters 280. The bands 114 a-f, (if provided) which have a known physical separation, and the catheter distal ends 284 can be moved close enough to the cutting area to support the cutting, but not so close as to get melted or damaged by the electrosurgical cutting procedure. Thus, an assembly, or system, is constructed for performing an electrosurgical procedure, and it may be assembled with reference to the markings 114a-f to ensure that the tips 284 of the catheters 280 are a predetermined distance from the cutting area.

Whether or not the opening is enlarged as described above, distal tips 284 of each catheter 280 can be advanced under visualization to a location proximal to the kinked denuded region of the guidewire, and indicia 114a-f on the guidewire can be used to maintain a predetermined spacing between the supporting catheters and the kinked denuded region of the guidewire to prevent damage to the supporting catheters. The method can further include activating the electrosurgical power source 800, and burning through the tissue of the myocardium using the kinked denuded portion 110b of the guidewire 100 to complete a cut through the myocardium, preferably while flushing at the same time with dextrose solution.

The disclosure also provides an electrosurgical system including a radio frequency power supply (such as that described in U.S. Pat. No. 6,296,636, which is incorporated by reference herein in its entirety for any purpose whatsoever) operably coupled to the electrically conductive core wire 115. Thus, the radio frequency power supply can be operably (and selectively) coupled to the electrically conductive core wire and to the second electrical conductor, as desired by way of a cable 600. Any suitable power level and duty cycle can be used in accordance with the disclosed embodiments. For example, continuous duty cycle (cutting) radiofrequency (“RF”) energy can be used, for example, at a power level between about 10 and 30 or 50 Watts, for example, or any increment therebetween of about one watt. The cuts can be made by applying power for between about one half of a second and about five seconds, or any increment therebetween of about one tenth of a second. The electrosurgery generator can be the Medtronic Force FX C Generator that achieves 5 W to 300 Watts of monopolar radiofrequency (RF) energy, for example.

Each of the supporting catheters can be made from a variety of materials, including multilayer polymeric extrusions, such as those described in U.S. Pat. No. 6,464,683 to Samuelson or U.S. Pat. No. 5,538,510 to Fontirroche, the disclosure of each being incorporated by reference herein in its entirety. Other structures are also possible, including single or multilayer tubes reinforced by braiding, such as metallic braiding material. Any of the catheters or guidewires disclosed herein or portions thereof can be provided with regions of varying or stepped-down stiffness with length using any of the techniques set forth in U.S. Pat. No. 7,785,318, which is incorporated by reference herein in its entirety for any purpose whatsoever.

The catheters disclosed herein can have a varied stiffness along their length, particularly in their distal regions by adjusting the cross-sectional dimensions of the material to impact stiffness and flexibility, while maintaining pushability, as well as the durometer of the material. Hardness/stiffness is described herein with reference to Shore hardness durometer (“D”) values. Shore hardness is measured with an apparatus known as a Durometer and consequently is also known as “Durometer hardness”. The hardness value is determined by the penetration of the Durometer indenter foot into the sample. The ASTM test method designation is ASTM D2240 00. For example, in some implementations, a more proximal region of the catheter can have a durometer of about 72D, an intermediate portion of the catheter (the proximal most 20-30 cm of the last 35 cm, for example that typically traverses an aortic arch) can have a durometer of about 55D, and the distal 5-10 cm of the catheter can have a durometer of about 35D.

Any surface of various components of the system described herein or portions thereof can be provided with one or more suitable lubricious coatings to facilitate procedures by reduction of frictional forces. Such coatings can include, for example, hydrophobic materials such as PolyTetraFluoroEthylene (“PTFE”) or silicone oil, or hydrophilic coatings such as Polyvinyl Pyrrolidone (“PVP”). Other coatings are also possible, including, echogenic materials, radiopaque materials and hydrogels, for example.

Implementations of the disclosed guidewires preferably include a sterile, single use device intended to cut soft tissue. References to dimensions and other specific information herein is intended to be illustrative and non-limiting. In one implementation, the disclosed guidewire has an outer diameter of 0.014″ and a working length of 260-300 cm. The proximal end of the disclosed guidewire, which has no patient contact, can be un-insulated to allow for connection to an electrosurgery generator.

Generally, cutting using the disclosed system can be performed by positioning the laceration (denuded mid-shaft) surface along the intended myocardial tissue, and applying traction on both free ends of the guidewire with the wire grippers 400 while simultaneously applying electrosurgery energy (typically 50-70 W) in short bursts, until the laceration is complete and the guidewire is free. The guidewire and catheters are removed.

The devices and methods disclosed herein can be used for other procedures in an as-is condition, or can be modified as needed to suit the particular procedure. This procedure for cutting the myocardium can be used in support of a variety of procedures. Likewise, while it can be appreciated that a monopolar cutting system is disclosed, in certain implementations, it is also possible to configure the system to operate in a bipolar configuration. During the step of myocardium laceration, the system can be configured to deliver energy to the myocardium with electrosurgical pads coupled to the patient to complete the circuit. When lacerating the myocardium or other structure with the bent denuded cutting wire, most of the energy is still dissipated in the patient.

In view of the many possible embodiments to which the principles of this disclosure may be applied, it should be recognized that the illustrated embodiments are only preferred examples of the disclosure and should not be taken as limiting the scope of the disclosure. Each and every patent and patent application referenced herein is expressly incorporated by reference herein in its entirety for any purpose whatsoever.

What is claimed is:

1. An electrosurgical guidewire, comprising:
a core wire having a proximal end, and a distal end and being defined by an outer surface between the proximal end and the distal end of the core wire, said core wire having a centerline that traverses the length of the core wire from the proximal end to the distal end of the core wire;
a dielectric coating disposed about the core wire, wherein the proximal end and distal end of the core wire are exposed and the proximal end is configured to be coupled to an electrosurgical generator;
a kink formed into the guidewire; and
an electrically exposed region located proximate the kink, the electrically exposed region being defined by a portion of the dielectric coating being absent from the surface of the core wire along an inner surface of at least one side of the kink, with the dielectric coating being left intact on an outer surface and an opposing inner surface of the kink.

2. The electrosurgical guidewire of claim 1, further comprising a radiopaque marker disposed over the core wire wherein the kink is proximate, adjacent, or at least partially within the radiopaque marker.

3. The electrosurgical guidewire of claim 2, wherein the dielectric coating is formed at least partially over the radiopaque marker.

4. The electrosurgical guidewire of claim 3, wherein the electrically exposed region of the kink is further defined by a portion of the radiopaque marker pattern being absent from the surface of the core wire along the inner surface of one side of the kink, with the radiopaque marker and dielectric coating being left intact on the outer surface and the opposing inner surface of the kink.

5. An electrosurgical system comprising
an electrosurgical generator;
an electrosurgical guidewire according to claim 1 coupled to the electrosurgical generator, wherein the electrically exposed region is disposed on only one side of the kink, and further wherein the electrosurgical generator is electrically coupled to one end of the guidewire and to the patient, wherein the electrosurgical system operates in a monopolar mode.

6. An electrosurgical system comprising
an electrosurgical generator;
an electrosurgical guidewire according to claim 1 coupled to the electrosurgical generator, wherein the electrically exposed region is disposed on both sides of the kink, and further wherein the electrosurgical generator is electrically coupled to both ends of the guidewire, wherein the electrosurgical system operates in a bipolar mode, and the current flows across tissue disposed in the kink during operation of the electrosurgical system.

7. The electrosurgical system of claim 6, wherein the electrically exposed region is defined by two discrete exposed areas separated by a fully insulated length of the guidewire in the region of the kink.

8. The electrosurgical system of claim 6, wherein the electrically exposed region defines at least one raised surface thereon to concentrate the electric field.

9. The electrosurgical system of claim 6, wherein the electrically exposed region includes a radiopaque electrically conductive coating.

10. The electrosurgical system of claim 9, wherein the radiopaque electrically conductive coating includes a metal or alloy of high electrical conductivity.

11. The electrosurgical system of claim 10, wherein the radiopaque electrically conductive coating includes gold.

12. The electrosurgical guidewire of claim 1, wherein the kink has a radius of curvature between about 1 mm and about 7 mm, between about 2 mm and about 5 mm, or between about 3 mm and about 4 mm.

13. The electrosurgical guidewire of claim 1, wherein the electrically exposed region has a length between about 1 mm and about 20 mm, a length between about 3 mm and about 6 mm, or a length between about 4 mm and about 8 mm.

14. The electrosurgical guidewire of claim 1, wherein the radiopaque marker is formed at least in part from a radiopaque metallic material deposited over the core wire.

15. The electrosurgical guidewire of claim 14, wherein the marker includes gold metallic material electroplated on the core wire.

16. The electrosurgical guidewire of claim 2, wherein the radiopaque marker includes an uneven surface configured to enhance its visual signature under fluoroscopy.

17. A method of performing a myocardial tissue cutting procedure, comprising:
directing a distal end of an electrosurgical guidewire according to claim 1 into the patient's vasculature through a passageway defined through myocardial tissue;
capturing the distal end of the electrosurgical guidewire with a snare catheter;
pulling the distal end of the electrosurgical guidewire out of the patient to externalize it alongside a proximal region of the electrosurgical guidewire;
advancing the electrosurgical guidewire until the electrically exposed region is in contact with the myocardial tissue within the passageway;
electrifying the electrosurgical guidewire; and
cutting the myocardial tissue by pulling the electrosurgical guidewire through the myocardial tissue.

18. The method of claim 17, further comprising directing at least one supporting catheter over a proximal portion of the externalized guidewire and a distal portion of the externalized guidewire until a distal tip of each said supporting catheter is located proximate the kink in the guidewire.

19. The method of claim 17, wherein the myocardial tissue includes a left ventricular outflow tract (LVOT) obstruction, and cutting the myocardial tissue enlarges the LVOT.

20. The method of claim 17, wherein the myocardial tissue includes ventricular tissue, and cutting the myocardial tissue enhances relaxation in hypertrophied myocardium.
